(19) United States
(12) Patent Application Publication
(10) Pub. No.: US 2021/0017676 A1
(43) Pub. Date: Jan. 21, 2021

(54) WEARABLES MADE OF RECYCLED MATERIALS AND A METHOD OF MAKING THE SAME

(71) Applicant: Joyce Greene Leslie, Highland Park, NJ (US)

(72) Inventor: Joyce Greene Leslie, Highland Park, NJ (US)

(21) Appl. No.: 16/947,019

(22) Filed: Jul. 15, 2020

Related U.S. Application Data
(60) Provisional application No. 62/874,060, filed on Jul. 15, 2019.

(51) Int. Cl. D02G 3/04; A61M 16/06; D01F 6/62; D01F 8/14; B29B 17/04

(52) U.S. Cl. CPC D02G 3/04; A61M 16/06; D10B 2501/00; D01F 8/14; B29B 17/0412; D01F 6/62

(57) ABSTRACT

A method of manufacturing a wearable material made from interweaving a primary yarn of recycled sheets of plasticized material and a water-proof companion yarn is provided. The wearable material may be rendered into articles of clothing as well as protective gear such as face masks or face mask covers.

### CROSS-REFERENCE TO RELATED APPLICATION

This application claims the benefit of priority of U.S. provisional application No. 62/874,060, filed 15 Jul. 2019, the contents of which are herein incorporated by reference.

### BACKGROUND OF THE INVENTION

The present invention relates to making wearables and, more particularly, wearables made of recycled materials and a method of making the same.

Those who care about their appearance detest when something they are wearing, for instance attractive clothes, are permanently ruin by a spot of spaghetti sauce or the like. Available protective napkins and bibs are ugly and do not work well, and thus not an option for these individuals.

Furthermore, during times of crisis, such a pandemic, when supplies and resources may be hard to come by in a pinch, the present invention provides a method of making protective gear, such as a face mask, from discarded materials, which may be easy to come by.

As can be seen, there is a need for a method of making a wearable material from recycled plasticized materials that protects the articles of clothing the clothing material is rendered from or protects the wearer when the wearable material takes the form of a mask for preventing the spread of disease or contagions.

### SUMMARY OF THE INVENTION

In one aspect of the present invention, a method of manufacturing a wearable material, the method includes the following: rendering one or more sheets of plasticized material into filament configurable as a primary yarn; selecting companion yarn; interweaving the primary yarn and companion yarn into a wearable material, wherein the one or more sheets of plasticized material is a plastic bag, wherein the companion yarn is waterproof, and wherein the companion yarn is a polartec material.

In another aspect of the present invention, a method of making a wearable from recycled plastic bag, the method includes the following: utilizing the method of claim 1, wherein the one or more sheets of plasticized material are one or more recycled plastic bags; and interweaving the wearable, wherein the wearable is a face mask or a face mask cover having a variegated surface.

These and other features, aspects and advantages of the present invention will become better understood with reference to the following drawings, description and claims.

### BRIEF DESCRIPTION OF THE DRAWINGS

FIG. 1 is a flowchart of an exemplary embodiment of the multi-step process embodied by the present invention;

FIG. 2 is a perspective schematic view of an exemplary embodiment of a step of a process embodied by the present invention;

FIG. 3 is a perspective schematic view of an exemplary embodiment of another step of the process embodied by the present invention;

FIG. 4 is a perspective schematic view of an exemplary embodiment of yet another step of the process embodied by the present invention;

FIG. 5 is a perspective schematic view of an exemplary embodiment of yet another step of the process embodied by the present invention; and

FIG. 6 is a perspective view of an exemplary embodiment of the product of the process embodied by present invention, shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

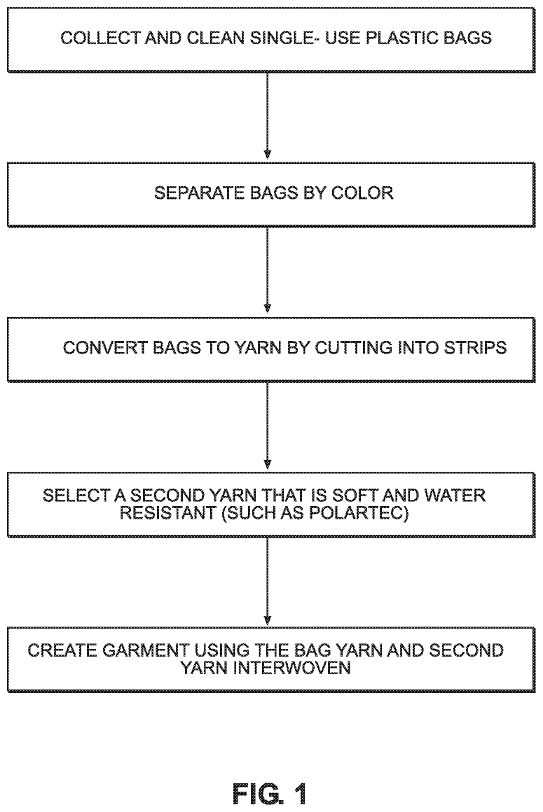

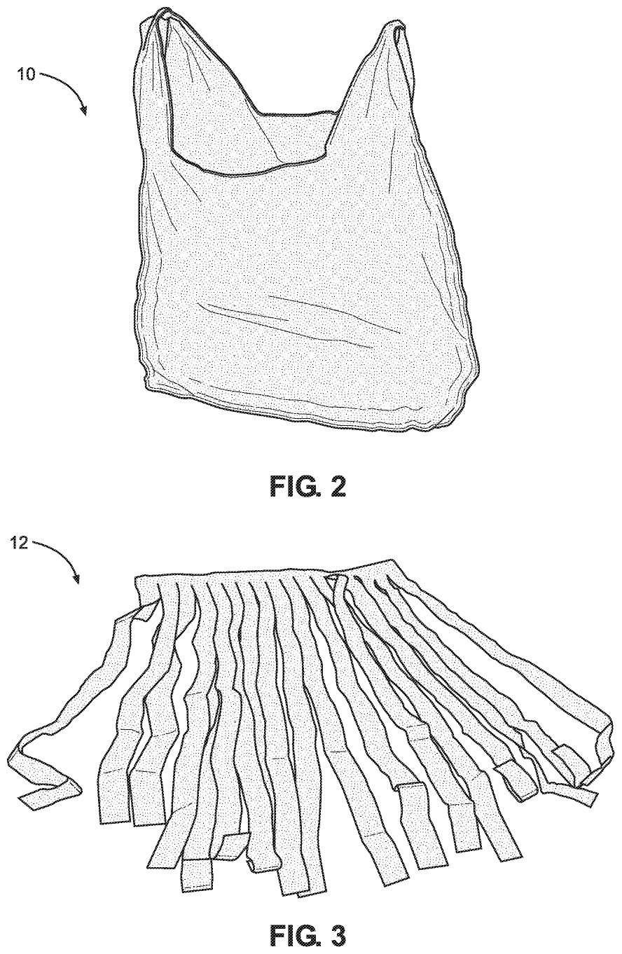

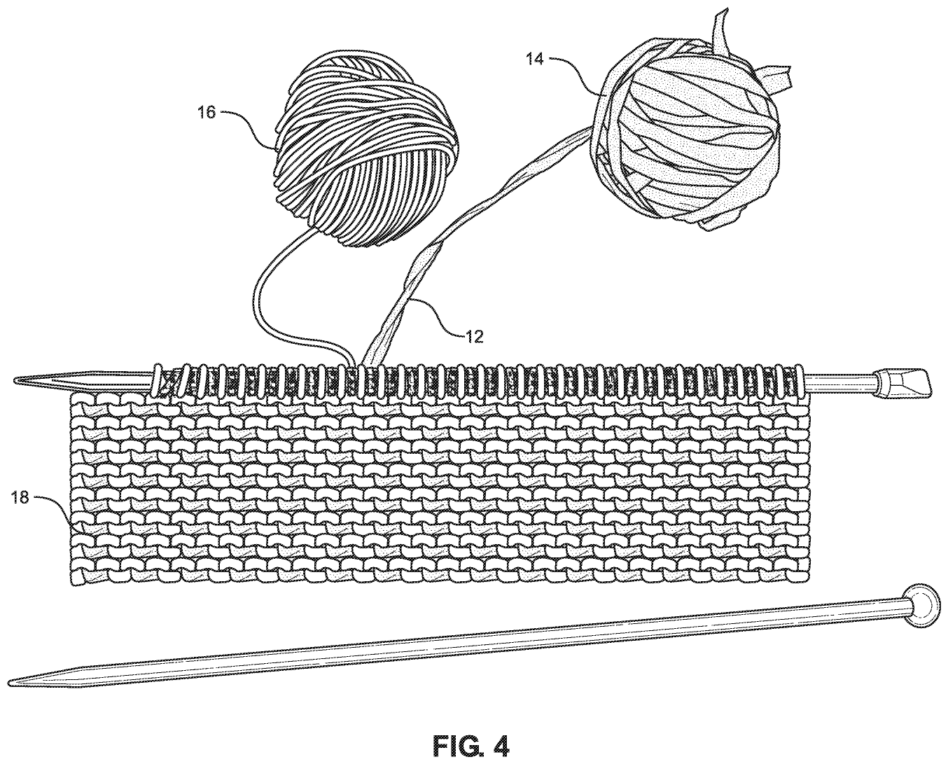

Referring to FIGS. 1 through 6, the present invention includes a method of making comfortable and protective clothing material and/or wearable material from which attractive articles of clothing and health-protective wearables can be rendered. The wearable material may include a primary yarn that is created from sheets of plasticized materials, including recycled plastic bags. The present invention is a method of making a wearable which is worn like clothing or gear and which is made from material that is water and oil resistant. The resulting garment/gear of the present invention may be fashionably casual or elegant yet comfortable and protective (obviating the need for a bib or an apron when around food products).

Additionally, in the face of the pandemic resulting from the novel coronavirus, the protective nature of the present invention is advantageous for additional reasons. With shortages due to COVID-19, the wearable material embodied in the present invention can be a lifesaver. According to John Hopkins Medicine, the fabric of any mask for protection from the novel coronavirus MUST have a pattern to it. The wearable material of the present invention affords users to create a mask (or other protective wearable) with a pattern from combination yarn and otherwise discarded plasticized material, which is a boon when factory-made masks and even standard clothing material may be hard to come by.

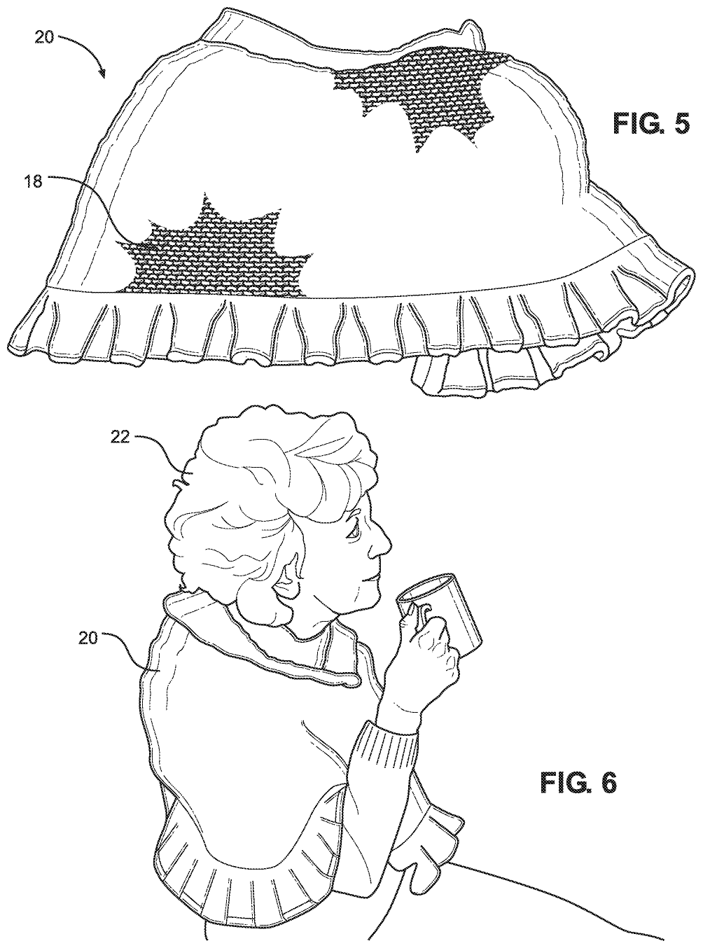

The garment of the present invention is made of a combination of novel materials that make it very protective and the basis for many fashionable designs. These features stem from the same source: a primary yarn created from discarded plasticized material, such as recycled grocery bags, combined with companion yarn to make a systemic wearable material yarn capable of being knitted into different designs. The companion yarn may include materials (such as polartec material) that soften and strengthen the plasticized material. Polartec material that is also a recycled polyethylene product. This novel wearable material can be used to knit a wide variety of designs; in certain embodiments an attractive wrap, as illustrated in FIGS. 5 and 6. This wrap is soft, pretty and water/oil proof. The wrap may be for the body, or when in need, smaller wraps can be fashioned as face mask to successfully reduce transmission of the novel coronavirus by preventing the passage of small droplets when the wearer or a nearby individual talks, sneezes or coughs.

Referring now to FIG. 1, the present invention may include the following components:
A. Obtain a supply of sheets of plasticized material 10 such as discarded plastic garbage, which are turned into a primary yarn 14, and separate by color is so desired.
B. Convert sheets of plasticized material into filament or strips of plasticized material 12.
C. Select a companion yarn 16 that is soft and water-resistant. (such as polartec material)
D. Once the strips of plasticized material 12 are cut (and rolled into a ball in certain embodiments), the wearable designed, the companion yarn chosen, and colors selected, the plasticized material yarn 14 and companion yarn 16 are used to knit into an interwoven systemic wearable material 18 of a selected design 20 for a wearer 22.

Step 1 may be cutting the raw material, the sheets of plasticized material 10, into filament or strips of plasticized material 12—i.e., primary yarn 14. This primary yarn 14 would most likely be then sorted by color and weight (as sheets of plasticized material 10 come in different colors and weights). Step 2 may be the selection of design of a companion yarn 16 to interweave with the primary yarn 14 to create a particular desired wearable 20. Component 3 may be the knitting and finishing of a particular wearable (be it a garment or a protective mask) 20 from the systemic wearable material created through the interweaving primary yarn and the companion yarn(s) 16.

The present invention can make use of many existing garment and wearable patterns and using the systemic wearable material 18—typically, where protective function is required. Note, also that the systemic wearable material 18 can be rendered through a combination of a plurality of companion yarns 16 of different qualities and weights to support different garment and wearable designs or portions thereof. The systemic wearable material 18 can be knitted into a single garment, a single wearable, or a specified set of squares, rectangles and triangles component shapes, wherein once the component shapes are knitted, they may be sewn together to produce the resulting garment or wearable.

The raw materials are sheets of plasticized material 10, typically plastic bags which are used by grocery and retail stores (but which are frequently then thrown away) as this resource seems to be mercilessly omnipresent. Thus, plastic bags have an initial use, after which they standardly become garbage that has a negative environmental impact. Through the present invention, these plastic bags may be collected or otherwise procured before production can begin. Other yarns and tools for cutting, knitting, finishing, etc. can be purchased.

Once the sheets of plasticized material 10 (e.g., plastic bags) are assembled, the method of turning the sheets of plasticized material 10 into the primary yarn 14 can be achieved by a person with an ordinary scissor or by a 3-D printer. Similarly, once the resulting wearable material can be knit into a garment or into geometric shapes by a person with knitting needles or by a knitting machine, etc. The design and requirements of the garments or wearables (e.g. the pattern or component shapes, the sizes, the quantity) occurs prior to knitting the components. The components may be assembled and/or produced mechanically or by hand.

The garment or wearable could be knitted like a traditional garment (one that is knitted from wool). It is also possible that ironing/heating the resulting knitted components of systemic wearable material improves the protective quality and makes additional design elements possible. The components of the protective systemic wearable material 18 could be used to create other garments/wearables 20 that need to be waterproof—like raincoats or hats. The use of systemic wearable material 18 can be rendered into a wide variety of objects 20 as well—like seat covers or car windshields. The primary yarn 14 can be knit into designs that are then melted into smaller shapes that could be used in design, art, or as components in protective garments. The rendering of the systemic wearable material 18 by way of the primary yarn 14 and the companion yarn 16 can be done through knitting, crocheting, sewing or any other known method of joining filaments of materials to form wearables or other objects.

A user may don the garment or wearable 20 to protect fine clothing in restaurants, at work, while traveling or at home. An attractive wrap can be worn over a delicate silk blouse while dining out. It might also be worn while traveling; it may protect the clothing worn by a driver or passenger from debris, make-up or coffee. It could also be worn by a patient in a hospital or rehabilitation unit—an attractive garment that does not humiliate the wearer but does provide protection from food and medicine that could soil clothing.

Another application of the protective systemic wearable material 18 is very current—the pandemic in 2020 has created a shortage of PPE, personal protective equipment. High quality masks are in short supply and people have been making them at home. Medical professionals report having to use their N95 masks for more than one day and sometimes up to a week. Different methods of keeping them protective have been publicized, but all agree that these masks were not intended to be reused.

However, the need to reuse these masks presented growing risks to professionals and may have contributed to rate of illness and death during the 2020 pandemic. Yet, using a reusable and washable cover for such masks would permit professionals to protect themselves and their standard masks (because they were protected) and to remain much safer when they do high risk work. Standard masks that are covered may indeed become safe to reuse.

The systemic wearable material 18 embodied in the present invention can be rendered into a mask cover wearable 20. The variegated surface that would be created by this mask cover will protect the N95 and lesser masks from many particulates in the environment and allow the underlying mask to itself be protected from environmental debris (biological and not biological), and lengthen the useful life of the underlying mask.

The recyclable cover for the mask does not have to be on at all times and could be employed when dealing with “high debris” events or entering “high debris” environments, as well as “high risk” situations, like an emergency room during a spike of Covid19 or another illness. It might also be useful during unexpected crowd situations, like an airport or during times of social unrest. The mask cover could hang around the neck of the wearer, just as other face masks do.

The weight of the systemic wearable material 18 can be selectively altered to make the mask cover lighter (through selection of the companion yarn 16). The mask cover would look like a mask but be a bit larger to fit over an ordinary mask, with and modified patterns to account for additional room (as it will cover an underlying mask). In certain embodiments, a mask cover made from the systemic wearable material 18 would be the same shape as current mask—but a bit deeper (for more room). Using the systemic wearable material (in certain embodiment a combination of plastic yarn and polartec material) manufacturers can create a mask cover that would protect wearers of ordinary masks in high risk situations like emergency rooms or airports.

It should be understood, of course, that the foregoing relates to exemplary embodiments of the invention and that modifications may be made without departing from the spirit and scope of the invention as set forth in the following claims.

What is claimed is:

1. A method of manufacturing a wearable material, the method comprising:
rendering one or more sheets of plasticized material into filament configurable as a primary yarn;
selecting companion yarn;
interweaving the primary yarn and companion yarn into a wearable material.

2. The method of claim 1, wherein the one or more sheets of plasticized material is a plastic bag.

3. The method of claim 1, wherein the companion yarn is waterproof.

4. The method of claim 1, wherein the companion yarn is a polartec material.

5. A method of making a wearable from recycled plastic bag, the method comprising:
utilizing the method of claim 1, wherein the one or more sheets of plasticized material are one or more recycled plastic bags; and
interweaving the wearable.

6. The method of claim 5, wherein the wearable is a face mask.

7. The method of claim 5, wherein the face mask has a variegated surface.

8. The method of claim 5, wherein the wearable is a face mask cover.
